(19) United States
(12) Patent Application Publication
(10) Pub. No.: US 2015/0099962 A1
(43) Pub. Date: Apr. 9, 2015

(54) Methods and Systems To Identify Phase-Locked High-Frequency Oscillations In The Brain

(71) Applicant: The Regents of the University of California, Oakland, CA (US)

(72) Inventors: Shennan Aibel Weiss, New York, NY (US); Catherine A. Schevon, New York, NY (US)

(21) Appl. No.: 14/507,432

(22) Filed: Oct. 6, 2014

Related U.S. Application Data
(60) Provisional application No. 61/887,658, filed on Oct. 7, 2013.

(51) Int. Cl. A61B 5/048; A61B 5/00; A61B 5/05

(52) U.S. Cl. CPC A61B 5/048; A61B 5/05; A61B 5/7282; A61B 5/7257
USPC 600/409; 600/544

(57) ABSTRACT

Method and device for the automatic identification of phase-locked high-frequency oscillations (PLHFO) to localize epileptogenic brain for neurosurgical intervention, including filtering brain signals into low frequency and high frequency oscillation (HFO) data streams. Applying ICA to the HFO data stream, transforming the data streams to produce an HFO instantaneous amplitude (HFOIA) and a low-frequency instantaneous phase (LFIP) data stream. Transforming the normalized HFOIA to produce an instantaneous phase of the normalized HFOIA. Determining a continuous or discrete PLHFO calculation that measures cross frequency coupling between the instantaneous phase of the low frequency data stream, and the instantaneous amplitude of the HFO data stream based at least in part on LFIP, raw or normalized HFOIA, and may include the instantaneous phase of normalized or raw HFOIA. Determining that at least a portion of the electrical signals from the brain display PLHFO if the PLHFO calculation is above a statistical threshold.

### CROSS-REFERENCE TO RELATED APPLICATIONS

This application claims priority to U.S. Provisional Application Ser. No. 61/887,658, filed on Oct. 7, 2013, which is incorporated by reference herein in its entirety.

### GRANT INFORMATION

This invention was made with government support under Grant Nos. K08 NS48871, ROI NS084142 and R25 10416928, awarded by the National Institute of Health. The U.S. government has certain rights in this invention.

### BACKGROUND

The disclosed subject matter relates to methods and systems for identifying electrical events generated by an epileptogenic brain during and between seizures. The detection and quantification of this activity can be used to determine the location of the epileptogenic brain, and its spatiotemporal spread during a seizure.

Medical refractory epilepsy refers to epilepsy that does not remit despite the use of multiple anti-epileptic medications, and affects 15 million people worldwide. The current treatment for medically refractory epilepsy consists of neurosurgical resection of the diseased cerebral cortex, or implantation of a medical device into this affected brain region. To determine the location of the surgery or device, electrical recordings from the scalp and brain are used to localize the location of seizures, and other abnormal brain activity.

The current standard of care for patients undergoing late stages of evaluation for epilepsy surgery is intracranial electroencephalogram (EEG) recordings from depth (i.e., sharp electrodes that penetrate into the brain) and subdural (i.e., brain surface) electrodes with multiple metal contacts, to identify the epileptogenic cerebral cortex. Analysis of the intracranial electroencephalogram recording is performed by a board-certified epileptologist and can be qualitative. Visual analysis of EEG recorded between seizures, i.e., inter-ictal recordings, can identify potentially epileptogenic brain by identifying the electrodes that detect discrete electrical events (called inter-ictal discharges). Visual analysis of EEG recorded during a seizure can identify epileptogenic brain by performing a qualitative determination of the electrode contacts detecting the earliest seizure activity.

Surgeries based on the approaches described above are not always effective. Only 30% of patients with non-lesional frontal lobe neocortical epilepsy will be seizure free 5 years after the operation. Difficulty localizing the epileptogenic region can be due to a failure to capture a seizure during intracranial EEG monitoring, or widespread seizures that are difficult to localize using the current qualitative analysis. Also, the site of respective surgery or device placement can be selected based on intracranial EEG inter-ictal recordings in the operating room alone, and without any prolonged monitoring.

High-frequency oscillations (HFOs) can be detected and isolated in the EEG recording using microwire, depth, subdural, epidural, or scalp electrodes. They can also be detected in the magnetoencephalogram. HFOs occurring during or between seizures can be isolated from background activity by visual inspection. This is labor-intensive and inter-reader reliability is questionable. Furthermore, this process is time-consuming and cannot be completed in real time. Also, distinguishing HFOs with pathological significance from physiological HFOs using visual inspection may not always be possible.

### SUMMARY

The disclosed subject matter provides systems and methods for identifying phase-locked high-frequency oscillations (PLHFO) in the brain. In an exemplary embodiment, a method of identifying brain electrical activity displaying PLHFO can include receiving electrical signals form the brain. The method can include filtering the electrical signals to produce an HFO data stream and a low-frequency data stream. The method can include applying independent component analysis to the HFO data stream and removing noise from the HFO data stream. The method can include transforming each of the HFO data stream and the low-frequency data stream to produce an HFO instantaneous amplitude and a low-frequency instantaneous phase. The method can include normalizing the HFO instantaneous amplitude to produce a normalized HFO instantaneous amplitude. The method can further include transforming the normalized HFO instantaneous amplitude to produce an instantaneous phase of the normalized HFO instantaneous amplitude. The method can include determining a continuous or discrete PLHFO calculation that measures cross frequency coupling between the instantaneous phase of the low frequency data stream, and the instantaneous amplitude of the HFO data stream at least in part on the low-frequency instantaneous phase, the raw or normalized HFO instantaneous amplitude, and may include the instantaneous phase of the normalized or raw HFO instantaneous amplitude. The method can include determining that at least a portion of the electrical signals from the brain display PLHFO if the PLHFO calculation is above a threshold.

In some embodiments, receiving electrical signals can include recording electrical signals with an electroencephalogram (EEG). In some embodiments recording can occur during a seizure. In some embodiments recording can occur between seizures. In some embodiments, receiving electrical signals from the brain can include receiving recordings from a magnetoencephalograph (MEG) device.

In particular embodiments, the method can include calculating the threshold for the continuous and discrete PLHFO measure based on statistical methods. The method can include supporting a therapeutic procedure based on the identified brain electrical activity displaying PLHFO. The therapeutic procedure can include surgical resection of a portion of the brain, targeted gene therapy of a portion of the brain, and implanting a therapeutic device in the brain. The method can include identifying a neurological or psychiatric illness associated with the PLHFO, including, but not limited to, one or more structural lesions to the brain, such as brain tumors.

In particular embodiments, receiving electrical signals from the brain can include receiving electrical signals from a plurality of recording electrodes. The method can include mapping the portion of the electrical signals from the brain displaying PLHFO in space and time. In some embodiments, filtering the electrical signal can include applying a bandpass filter. In some embodiments, transforming the data streams can include transforming the data streams with a Hilbert transform.

The accompanying drawings, which are incorporated in and constitute part of this specification, are included to illustrate and provide a further understanding of the method and system of the disclosed subject matter. Together with the description, the drawings serve to explain the principles of the disclosed subject matter.

### BRIEF DESCRIPTION OF THE FIGURES

FIG. 1 illustrates the concept of cross frequency coupling.

FIGS. 2A and 2B shows intracranial EEG recordings from patients during a seizure.

FIGS. 3A through 3B show both neuronal action potentials, EEG phase related neuron activity amplitude, and EEG phase related high frequency oscillation amplitude for epileptogenic and healthy brain.

FIG. 4 provides a method for calculating the PLHFO metric time series.

FIGS. 5A and 5B show seizures recorded from an intracranial electrode in epileptogenic and healthy brain, and corresponding PLHFO time series.

FIG. 6 provides a method for determining the PLHFO threshold.

FIGS. 7A through 7C illustrate the implementation of a method for calculating the PLHFO to determine the location of epileptogenic brain.

FIG. 8 illustrates the implementation of a method for calculating the PLHFO to map the spread of PLHFOs in space and in time.

FIGS. 9A and 9B provide data showing that resection of brain that generates phase PLHFOs results in successful epilepsy surgery.

FIG. 10 provides a method for identification of discrete inter-ictal HFOs and the calculation of corresponding HFO phasors for each discrete event.

FIG. 11 provides a method for optimization of HFO detection and the tallying of the number of PLHFO events.

FIG. 12 provides a second method for optimization of HFO detection and the tallying of the number of PLHFO events.

FIGS. 13A through 13C provide discrete HFO band events detected during an inter-ictal recording from two different recording electrodes in epileptogenic brain, the corresponding EEG band recordings for these events, and the resulting phase locked population of the HFO phasors.

FIG. 14 illustrates the population of HFO phasors isolated from an inter-ictal epoch in the epileptogenic brain and healthy brain.

FIG. 15 illustrates inter-ictal discharges isolated from an inter-ictal epoch from a recording electrode in epileptogenic brain.

FIG. 16 provides a method for detection of inter-ictal discharges in the inter-ictal EEG.

FIG. 17 illustrates implementation of the method of the disclosed subject matter in real time to localize epileptogenic brain.

FIG. 18 provides data that resection of brain generating both inter-ictal discharges and PLHFO correlates with successful epilepsy surgery.

FIG. 19 provides a block diagram of a computer system.

FIG. 20 provides a diagnostic algorithm using the SOZ and PLHG metrics, and resulting surgical outcome classification.

FIG. 21 table of patient characteristics

FIGS. 22A through 22E provide data demonstrating the delayed onset and limited extent of amplitude-modulated high gamma activity.

### DETAILED DESCRIPTION

The methods and systems presented herein can be used for identifying phase-locked high-frequency oscillations (PLHFO) in the brain.

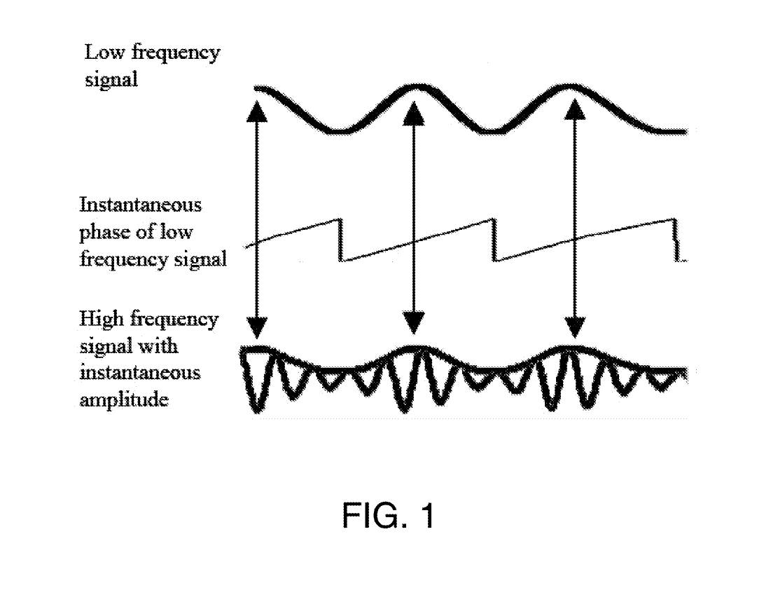

FIG. 1 shows, for the purpose of illustration and not limitation, the concept of cross frequency coupling. Cross frequency coupling occurs when the phase of one frequency (for example, a low frequency band) modulates the amplitude of a different frequency band (for example, a high frequency band). The maxima of the high frequency amplitude occur at the same phase of the low frequency signal (illustrated by vertical arrows). The methods and systems presented herein can detect and quantify high-frequency oscillations (HFO), for example, with a frequency between 50 and 600 HZ, including subsets of frequency bands within the range, that are cross frequency coupled with the phase of the EEG in the low frequency bands (delta-low gamma), including subsets of frequency bands within the range. In addition, the device can detect inter-ictal discharges. As used herein, the term EEG band refers to delta-low gamma frequency bands, since delta-low gamma bands are the traditional frequency bands of the EEG.

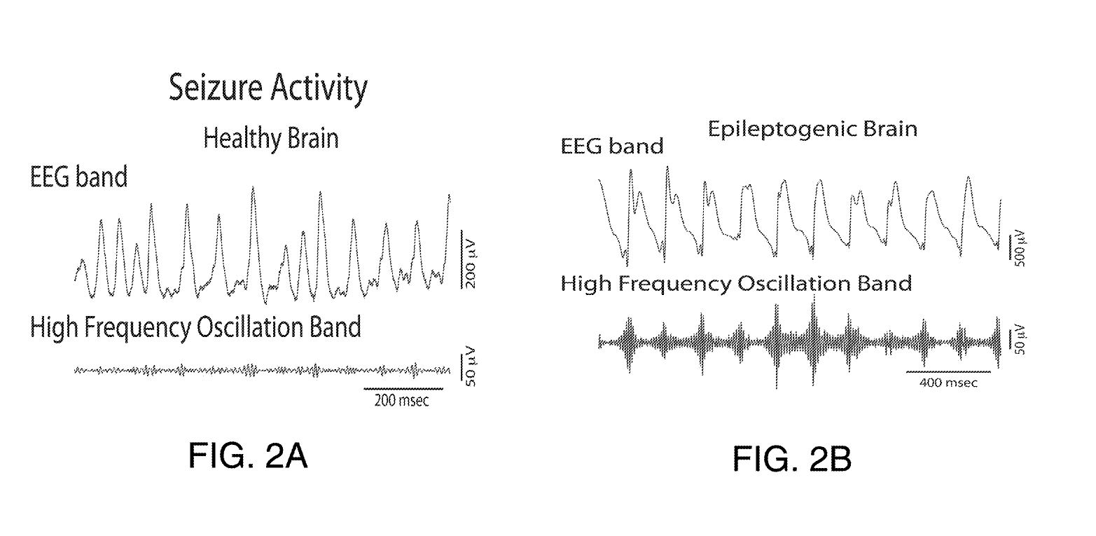

The high-frequency oscillation band can be isolated from the broadband EEG with bandpass digital filtering. The low-frequency EEG band can be isolated from the broadband EEG using a bandpass filter, for example 4-30 HZ. During a seizure HFO band amplitude can be modulated by the phase of the low-frequency EEG in epileptic brain regions, but not outside these regions. For example, FIG. 2A illustrates the EEG band and the HFO band of a healthy brain. There is no amplitude modulation in FIG. 2A. FIG. 2B illustrates the intracranial EEG recordings from a patient during a seizure that demonstrate that in an epileptogenic brain the amplitude of the HFO are cross frequency coupled with the phase of the EEG band.

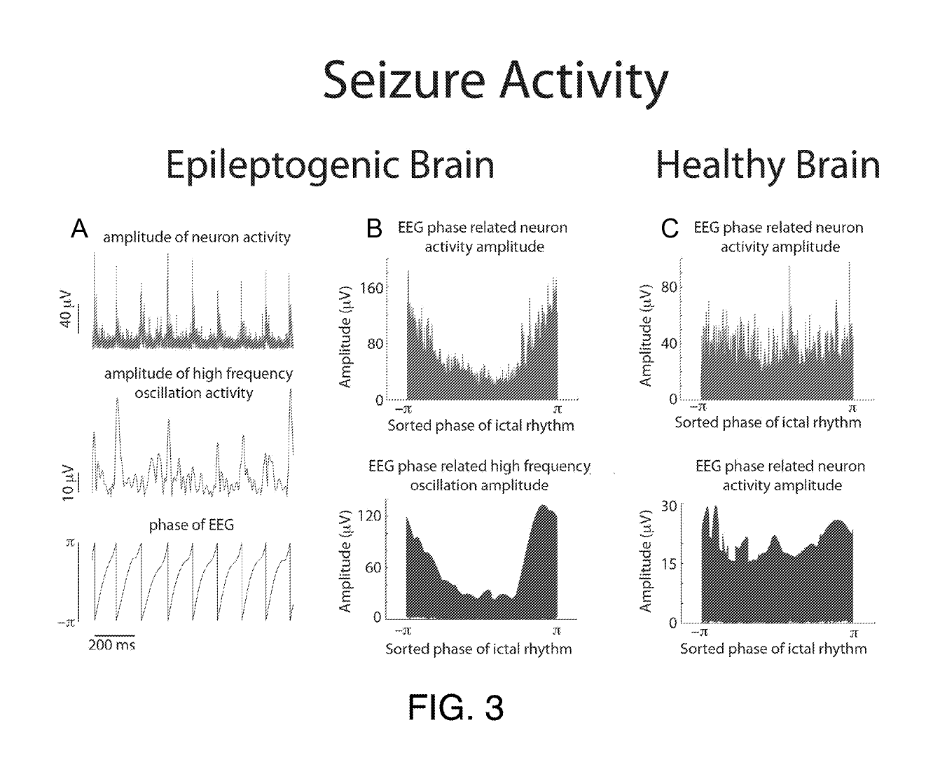

Furthermore, FIG. 3 shows, for the purpose of illustration and not limitation, that in epileptogenic brain regions during a seizure, both the occurrence of neuronal spiking and the amplitude of high frequency oscillations are cross frequency coupled to the phase of the EEG band. However, this cross frequency coupling is not evidence in healthy brain during a seizure.

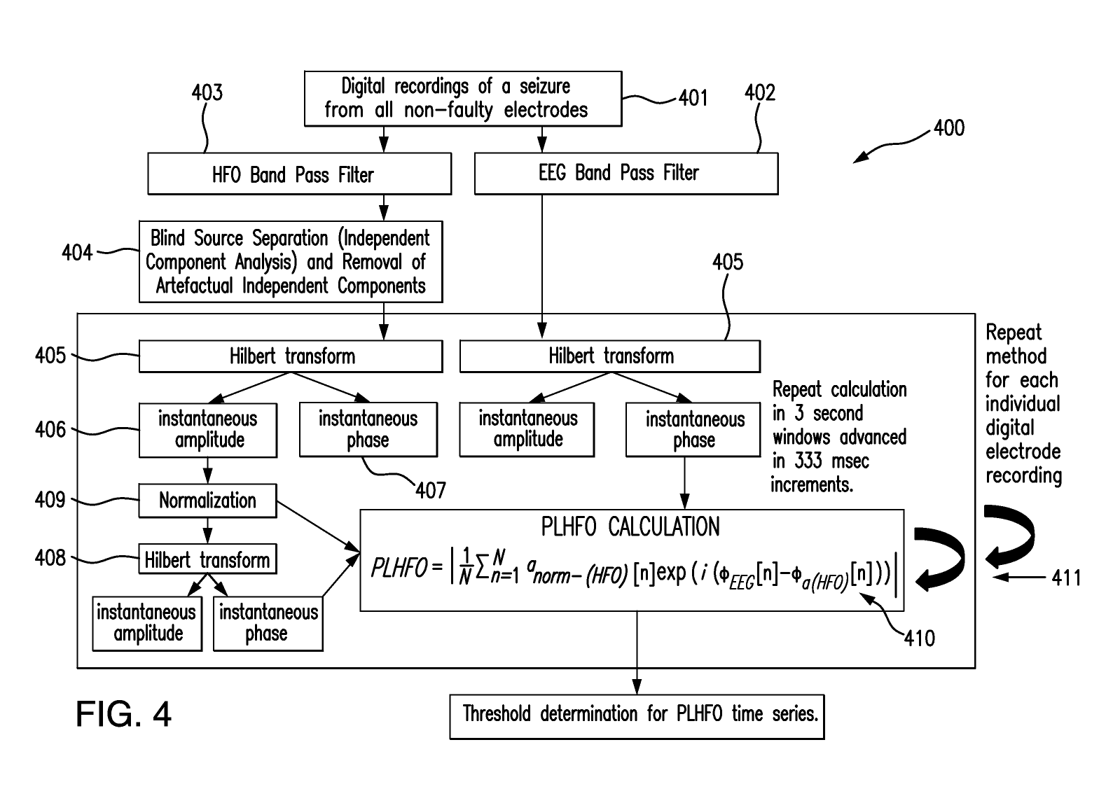

FIG. 4 shows, for the purpose of illustration and not limitation, a method (400) for identifying phase-locked high-frequency oscillations (PLHFO) in the brain in accordance with the disclosed subject matter. The PLHFO metric can be calculated for every valid recording electrode using the method (400) shown in FIG. 4. The PLHFO metric can be calculated by bandpass filtering (402, 403), using for example a high order digital finite impulse response filter, the raw EEG signal (401) into HFO band (403) and traditional EEG band (402) data streams. To remove noise from the HFO band pass filtered data stream, a blind source separation using independent component analysis (ICA) can be applied to the HFO band pass filtered recording of the seizure from all valid electrodes (404). The ICA algorithm can be FastICA, Infomax, or other suitable ICA algorithms. If Infomax is used, the first three independent components can contain the high frequency noise and can be removed from the time series for all the recordings from valid channels. A Hilbert transform (405) can be applied to both data streams and can result in the analytic signal z[n]=a[n]exp(i*phi[n]), where a[n] is the instantaneous amplitude of y[t] (406), and phi[n] is the instantaneous phase of y[t] (407). A second Hilbert transform (408) can be applied to the instantaneous amplitude of the HFO. In addition, the amplitude of the HFO band can be normalized (409). For example, the HFO band can be normalized by dividing by the time series values by the mean of the HFO amplitude during a 30 second inter-ictal epoch recorded from the same electrode. The PLHFO measurement can be calculated using Equation 1 (410).

$$PLHFO = \vert \frac{1}{N} {\sum }_{n = 1}^{N} {a}_{norm\_HFO} [ n ] exp ( i ( {φ}_{EEG} [ n ] - {φ}_{aHFO} [ n ] ) ) \vert \qquad Equation 1$$

Where a_{norm-HFO} is the instantaneous normalized amplitude of the HFO band, φ_{EEG} is the instantaneous phase of the EEG band, and φ_{aHFO} is the instantaneous phase of the instantaneous amplitude of the HFO band time series, and n is time.

Equation 1 scales instantaneous normalized high-frequency oscillation amplitude by the vector defined by the Phase Locking Value (PLV), shown in Equation 2, in the complex plane prior to calculating the net mean vector.

$$PLV = \vert \frac{1}{N} {\sum }_{n = 1}^{N} exp ( i ( {φ}_{EEG} [ n ] - {φ}_{aHFO} [ n ] ) ) \vert \qquad Equation 2$$

The result can be a direct measure of the HFO band amplitude that is phase locked to the traditional EEG rhythm. To calculate the PLHFO time series a sliding window method can be applied (411). Using a discrete window duration, the window can be advanced in set increments along the derived time series. The PLHFO time series for each valid electrode can be calculated. The PLHFO metric can capture the transient increases in HFO amplitude in their low-frequency context that is indicative of an epileptogenic brain region.

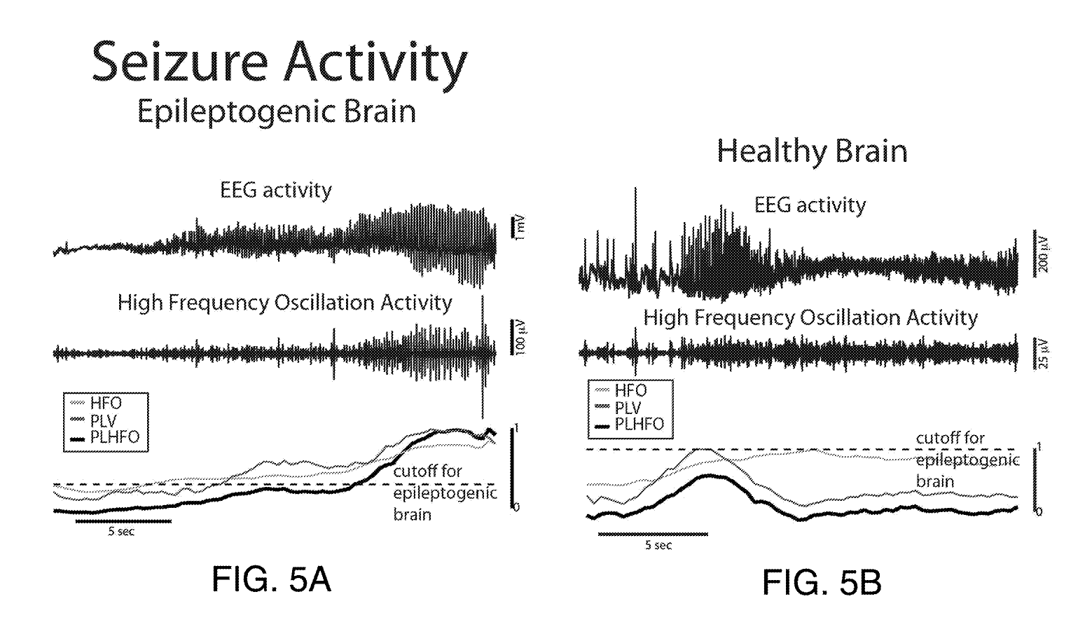

For example, FIG. 5 illustrates seizure recorded from an intracranial electrode in epileptogenic and healthy brain, as well as corresponding PLHFO time series. FIG. 5A illustrates an epileptogenic brain, and the PLHFO is above a threshold. FIG. 5B illustrates a healthy brain, and the PLHFO is below a threshold.

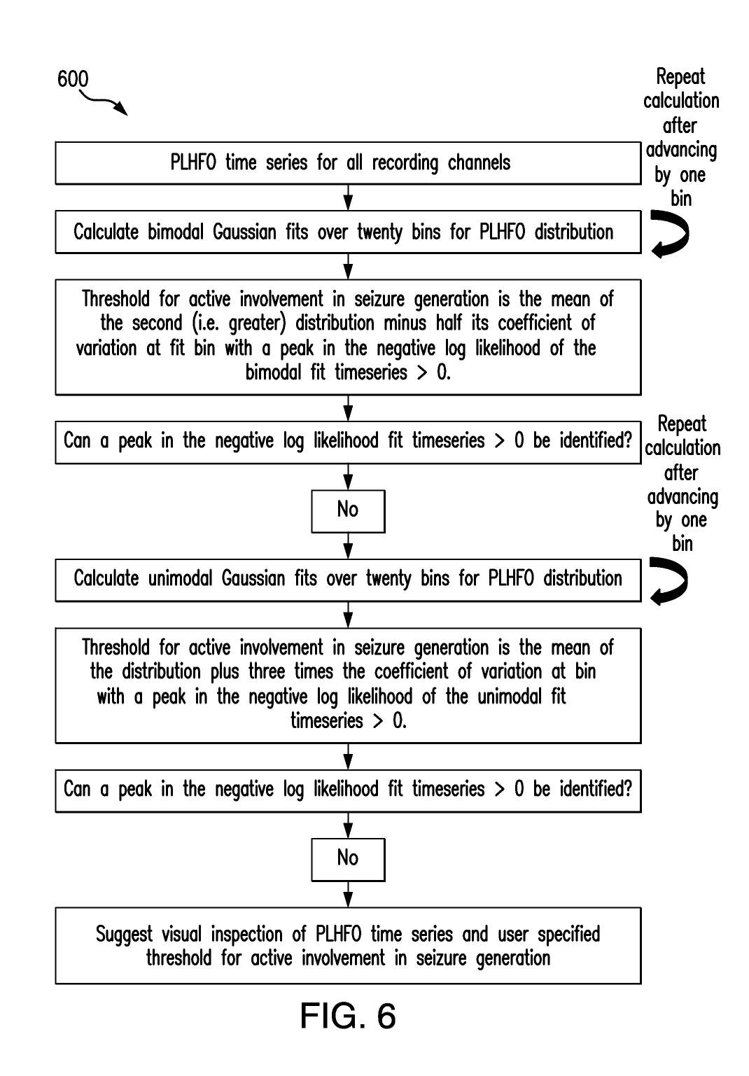

FIG. 6 shows, for the purpose of illustration and not limitation, a method (600) for determining the thresholds for defining epileptic brain on the basis of the PLHFO metric. Unimodal and bimodal Gaussian fits, also referred to as mixed Gaussian models, can be repeatedly calculated for the distribution of PLHFO values for all electrodes over twenty bins or more using a sliding window that can be advanced in steps of a single bin. If the first peak of the negative log likelihood of the bimodal fit is greater than zero, then the threshold for recruitment into the seizure can be derived as the mean of the second distribution−0.5*coefficient of variation of the second distribution. If the peaks of the negative log likelihood of the bimodal fit is less than zero across all time points of the seizure the distribution of phase locked high gamma (PLHG) can be assumed to follow a unimodal Gaussian distribution. The threshold for recruitment into the seizure core can be calculated at the time point demonstrating the first peak in the negative log likelihood of the unimodal fit that is greater than zero, and the threshold can be defined as the mean of the distribution+3*coefficient of variation. In the subset of recordings in which the peak of the negative log likelihood is not greater than zero, the threshold can be manually determined.

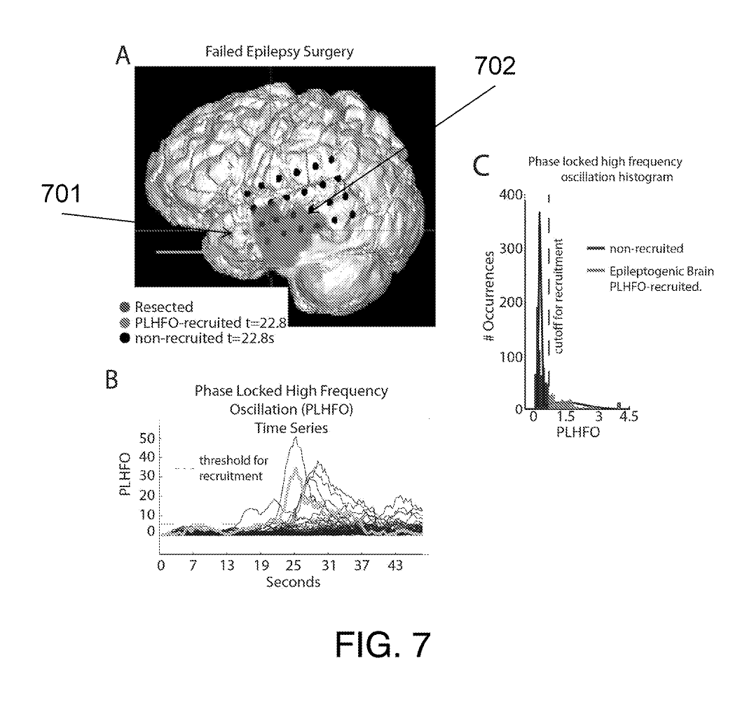

FIG. 7 shows, for the purpose of illustration and not limitation, the results of measurements and calculations taken from an epileptogenic brain, which received a failed resection surgery. FIG. 7B shows the PLHFO time series during a seizure for all the intracranial recording electrodes. The electrodes with a corresponding PLHFO time series that exceeds the threshold defined using the algorithm described above at 22.8 seconds are colored grey (701) in the 3D reconstruction of this patient's brain, illustrated in FIG. 7A. As shown in FIG. 7A, the electrodes associated with the PLHFO that exceeds the threshold are located outside the margins of the resection (702). The patient's epilepsy surgery failed. FIG. 7C illustrates the histogram of PLHFO values used in the algorithm described above and in FIG. 6 to define the threshold for a PLHFO recruited electrode or epileptogenic brain region. The distribution is bimodal, and the light colored PLHFO values represent PLHFO recruited electrodes.

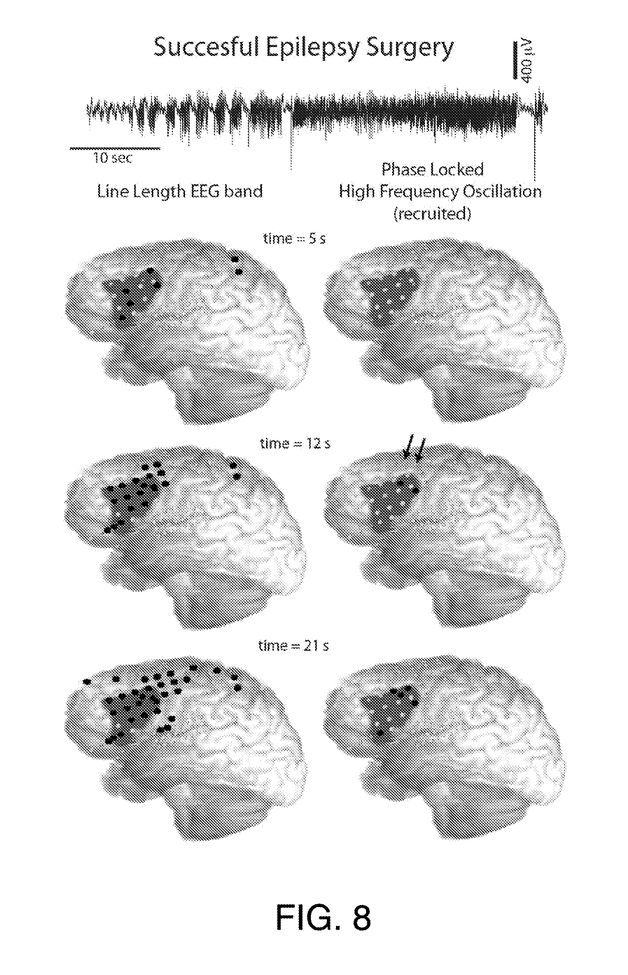

FIG. 8 shows, for the purpose of illustration and not limitation, the results of measurements and calculations taken from an epileptogenic brain, which received a successful resection surgery. THE PLHFO time series values from all the recording electrodes are projected onto a 3D reconstruction of the brain at different time points, thereby mapping the initiation and spread of the PLHFO recruited electrodes in space and time. The PLHFO electrodes were located exclusively within the resection cavity and the patient had a successful epilepsy surgery. Also, the PLHFO measure was more specific and spread was slower than an EEG band line length based indicator of seizure initiation and spread.

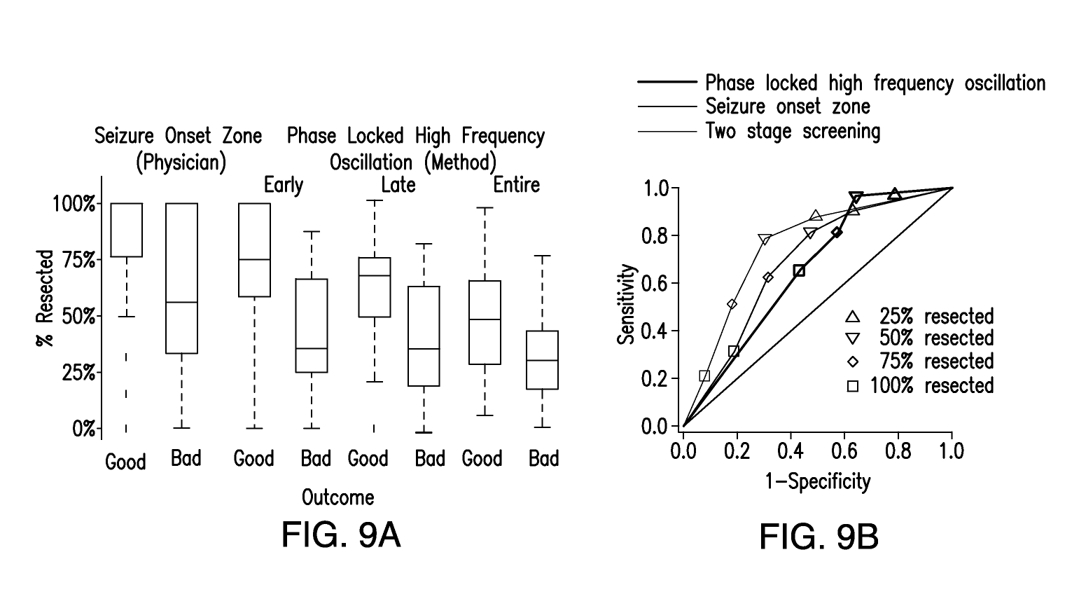

As shown in FIG. 9, patients that had resections that included more of the PLHFO-recruited electrodes were more likely to have successful surgeries. This was particularly true when the first four of the PLHFO recruited electrodes (early) were in the resection cavity. The measure of the percent of the first four PLHFO recruited electrodes that were resected was superior to the percent of the epileptologist defined seizure onset zone resected in accurately classifying patients with successful epilepsy surgeries from those with failed surgeries. In addition, combining the epileptologist defined seizure onset zone with the first four PLHFO recruited electrodes in a two stage screening resulted in the most accurate measure. Thus, the methods and systems disclosed herein can automatically identify epileptogenic regions.

Inter-ictal EEG can be recorded between seizures when the patient is awake, asleep, comatose, or anesthetized. Inter-ictal discharges and HFOs in the inter-ictal EEG can be used to determine epileptogenic brain regions.

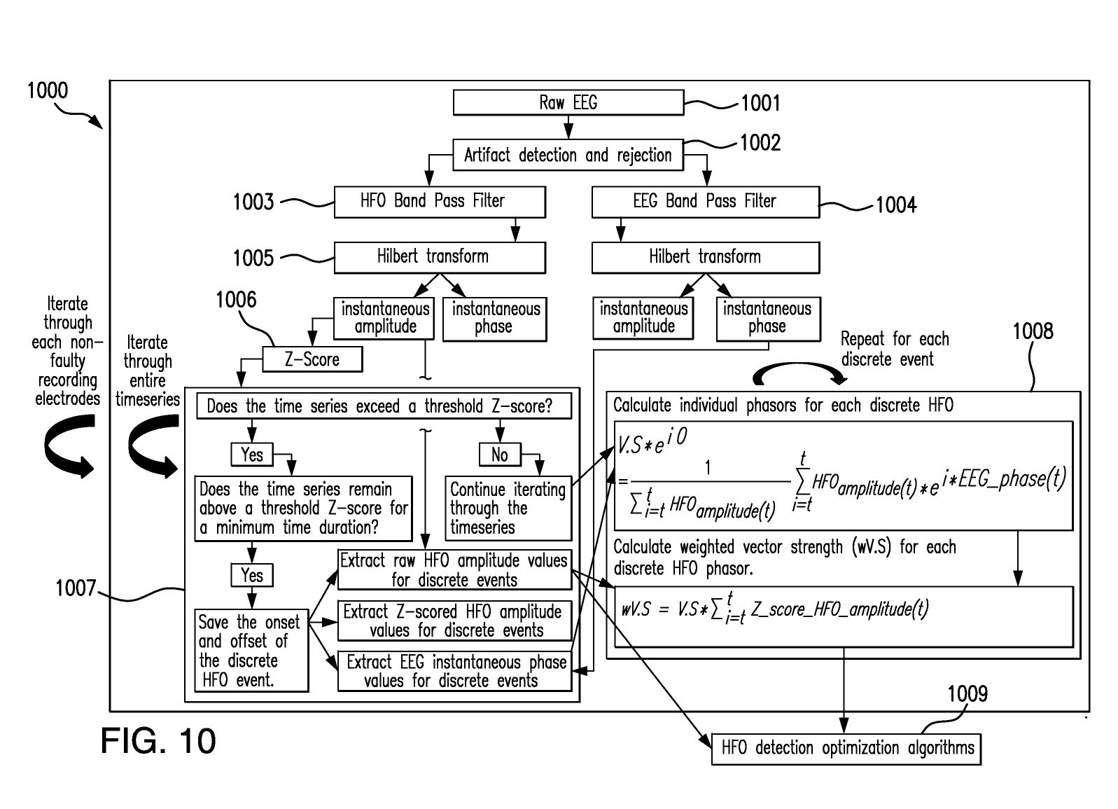

FIG. 10 shows, for the purpose of illustration and not limitation, a method (1000) for isolating discrete HFOs from the inter-ictal EEG and calculating individual phasors for each discrete HFO. The raw digital recording (1001) from each sensor can be split into a HFO band pass filtered stream (1003) and an EEG ban pass filtered stream (1004). For example, the streams can be filtered using a high order digital finite impulse response filter. If the raw signal exhibits obvious artifacts, then the data segment can be excluded (1002). A Hilbert transform (1005) can be applied to both the HFO band and the EEG band data streams to calculate the instantaneous amplitude and phase time series for each data stream. HFO band amplitude time series can be normalized, for example, by z-score (1006). A high sensitivity and low specificity detector can determine the onset and offset of discrete and continuous HFO events that exceed a set threshold z-score value for a predetermined duration of time (1007). The onset and offset of all the events can be recorded over the course of the entire inter-ictal epoch. The amplitude, normalized amplitude, and corresponding EEG band instantaneous phase vector can be stored for each discrete HFO event. A phasor can be calculated (1008) for each discrete continuous HFO event using Equation 3.

$$V \cdot S * {e}^{i θ} = \frac{1}{{\sum }_{i = t}^{t} {HFO}_{amplitude ( t )}} {\sum }_{i = t}^{t} {HFO}_{amplitude ( t ) * {e}^{i * EEG\_phase ( t )}} \qquad Equation 3$$

Where t is time. The phasor includes an individual HFO vector strength (V.S) between 0 and 1, and a mean phase angel θ.

Additionally, a HFO individual weighted vector strength (wVS) can be calculated for each discrete HFO phasor using Equation 4.

wV·S=V·S*Σᵢ₌ₜ^{t}Z_score_HFO_amplitude(t) Equation 4

All the discrete HFO events and corresponding HFO phasors can be identified and calculated for all active recording sensors.

Since HFOs, and corresponding phasors, can be detected using a low specificity detector, an optimization algorithm can be used to determine a normalized HFO amplitude threshold that can redefine the number of identified HFO events and phasors by excluding the events with a mean normalized HFO amplitude that does not meet the threshold (1009). The identification of the normalized amplitude threshold can be determined for each individual sensor independently, and using two independent algorithms. Both algorithms can be based on the premise that valid and pathologic HFOs are most likely to have corresponding phasors that are as a population statistically phase locked.

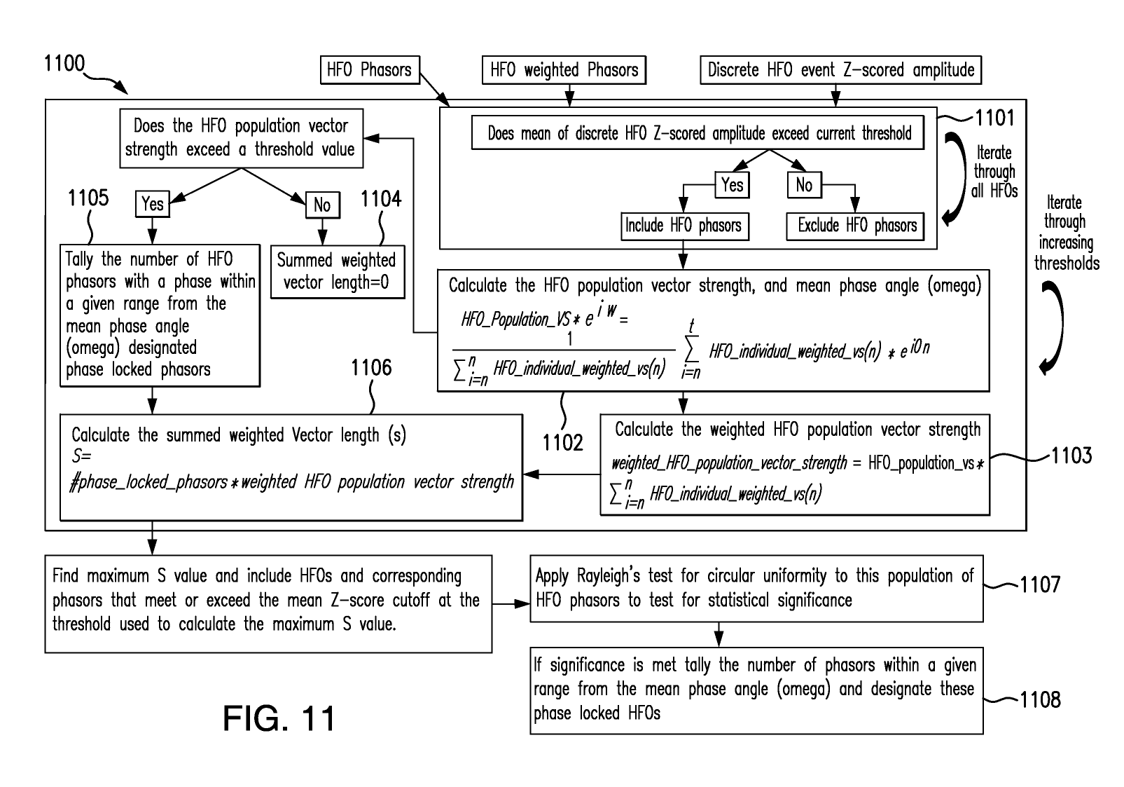

FIG. 11 shows, for the purpose of illustration and not limitation, a method (1100) to optimize the detection of inter-ictal HFOs and tally the number of phase locked HFOs. The method can find the optimal normalized HFO amplitude threshold by calculating a measure called the summed weighted vector strength (S) at incrementally increasing normalized HFO amplitude thresholds. The normalized HFO amplitude threshold that results in the maximum S value can be designated the optimal threshold.

The method can include excluding the HFOs, and corresponding phasors, that have a mean normalized amplitude less than the current threshold (1101). The method can include calculating the vector strength and mean phase angle of the population of valid HFOs using Equation 5 (1102).

$$HFO\_Population \_VS * {e}^{i ω} = \frac{1}{{\sum }_{i = n}^{n} HFO\_individual \_weighted \_vs ( n )} {\sum }_{i = n}^{n} HFO\_individual \_weighted \_vs ( n ) * {e}^{{iθ}_{n}} \qquad Equation 5$$

Where n refers to each discrete HFO in the valid population. HFO_population_VS is between 0 and 1, and omega (ω) is the mean phase angle of the population of valid phasors.

Additionally, on the basis of the wV.S values for each of the individual valid phasors, the weighted HFO population vector strength can be calculated with Equation 6 (1103).

weighted_HFO_population_vector_strength=HFO_population_{—}vs*Σᵢ₌ₙⁿHFO_individual_weighted_{—}vs(n) Equation 6

If the HFO_Population_VS does not exceed a threshold value, the summed_weighted_vector_strength (S) can be set to zero (1104). Otherwise, the number of valid HFO phasors with a mean phase angle θ within a predetermined phase range of ω (the mean phase angle of the population of valid phasors) can be tallied as phase locked phasors (1105). The summed weighted vector strength (S) can be calculated with Equation 7 (1106).

S=#phase_locked_phasors*weighted HFO population vector strength Equation 7

The valid HFO events at the optimal mean normalized HFO amplitude cutoff can be tested for statistically significant phase locking, for example, using Rayleigh's test for circular non-uniformity (1107). If the population of valid HFO phasors are statistically phase locked, the number of valid HFO phasors with a mean phase angle θ within a predetermined phase range of ω (the mean phase angle of the population of valid phasors) can be tallied as the number of phase locked HFOs (1108). If statistical significance is not met, the number of phase locked HFOs can be set to zero.

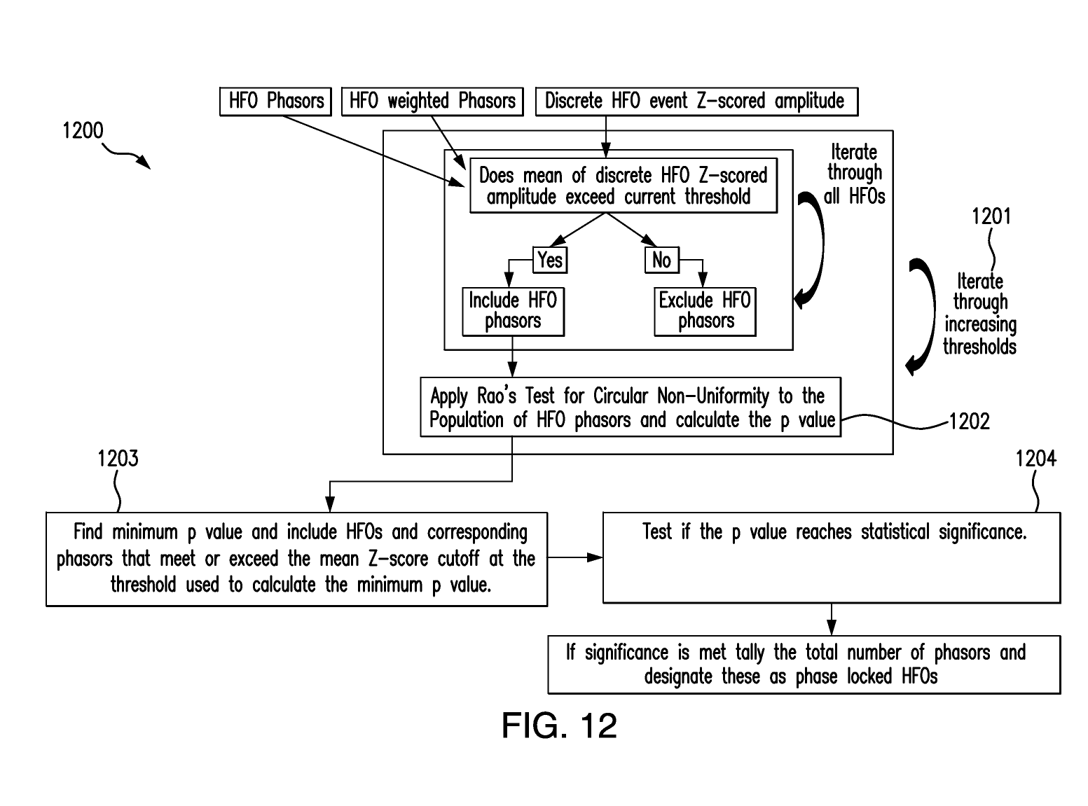

FIG. 12 shows, for the purpose of illustration and not limitation, a method (1200) for optimizing the detection of inter-ictal HFOs and tallying the number of phase locked HFOs. The method of FIG. 12 can be used because the use of Rayleigh's test (as used in the method 1100) is based on a unimodal assumption and the population of HFO phasors can be bimodally distributed. In the method of FIG. 12, the threshold value for excluding HFOs on the basis of mean normalized HFO amplitude can be iteratively increased (1201). At each threshold value Rao's test for circular non-uniformity can be applied to the population of valid phasors (1202). The optimal normalized HFO amplitude threshold can be determined as that which resulted in the lowest p value resulting from Rao's test (1203). The valid HFO phasors at this optimal threshold are tallied as phase locked HFOs if the p value resulting from Rao's test applied to this population of phasors meets a predetermined level of significance (1204). Otherwise, the number of phase locked HFOs can be set to zero.

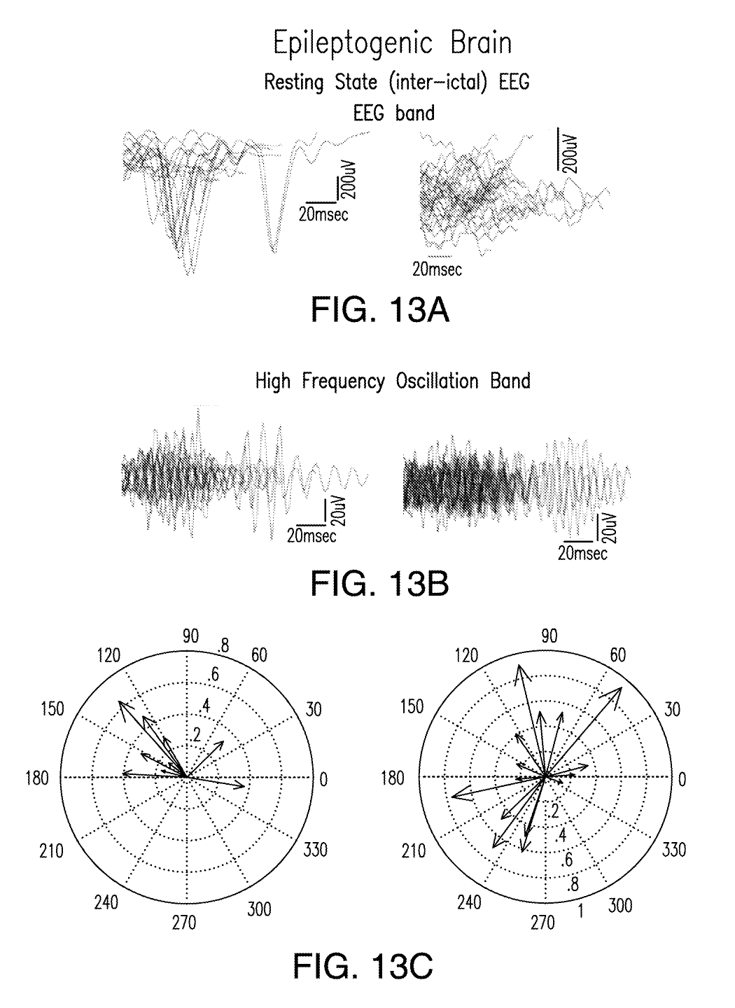

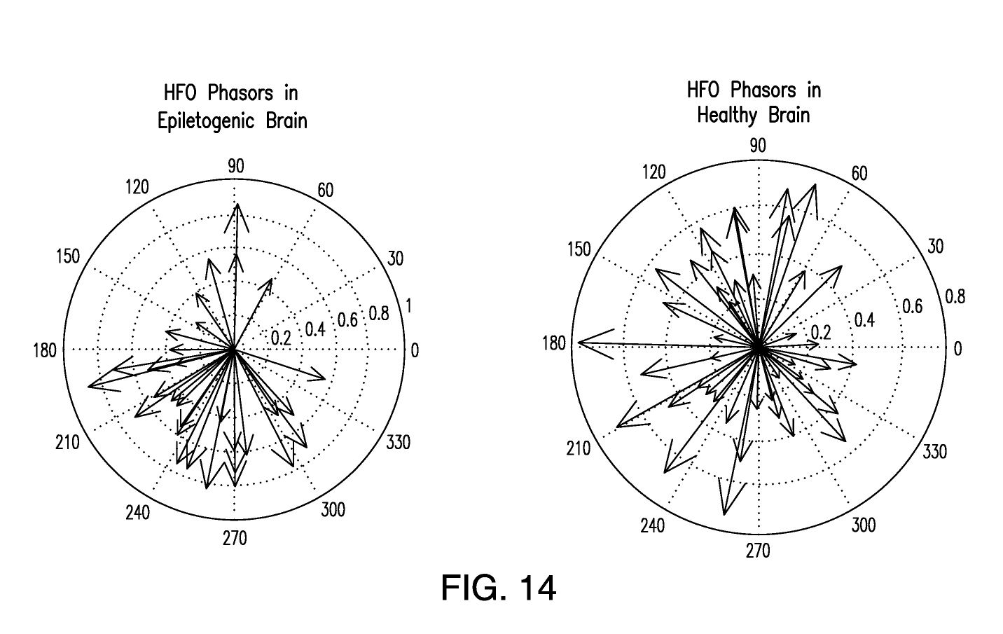

FIG. 13 shows, for the purpose of illustration and not limitation, the EEG band (top), HFO band (middle), and corresponding phasors (bottom) of a population of HFOs detected during an inter-ictal epoch from electrodes in epileptogenic brain. The example on the left shows a clear unimodal distribution of HFO phasors, while the example on the right shows a clear bimodal distribution. In contrast, the HFO phasors in a healthy brain (for example as shown in FIG. 14 on the right) are more likely to be uniformly distributed.

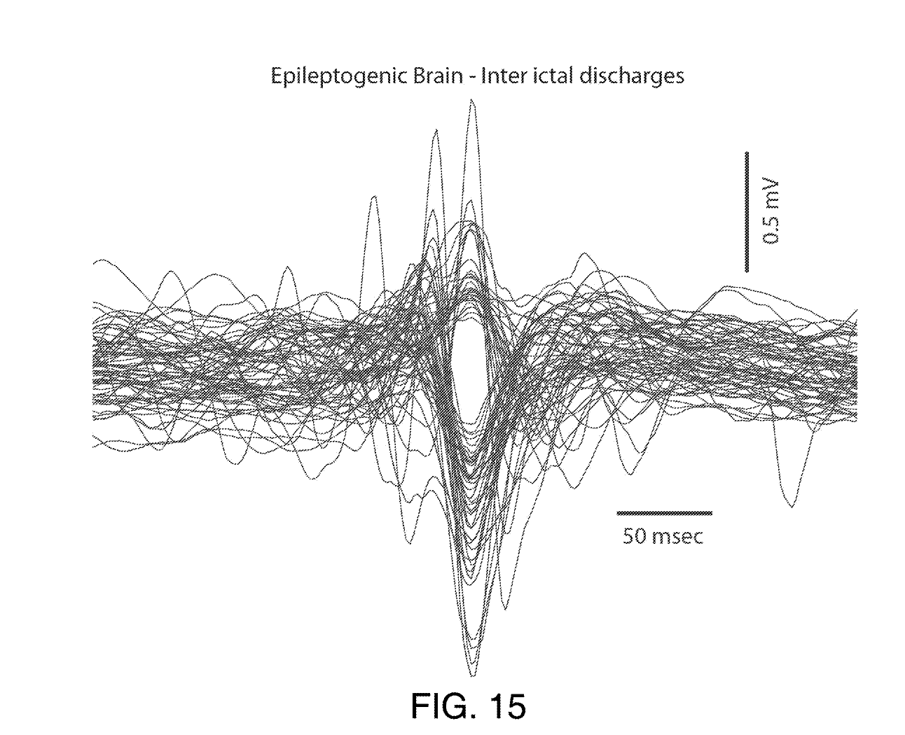

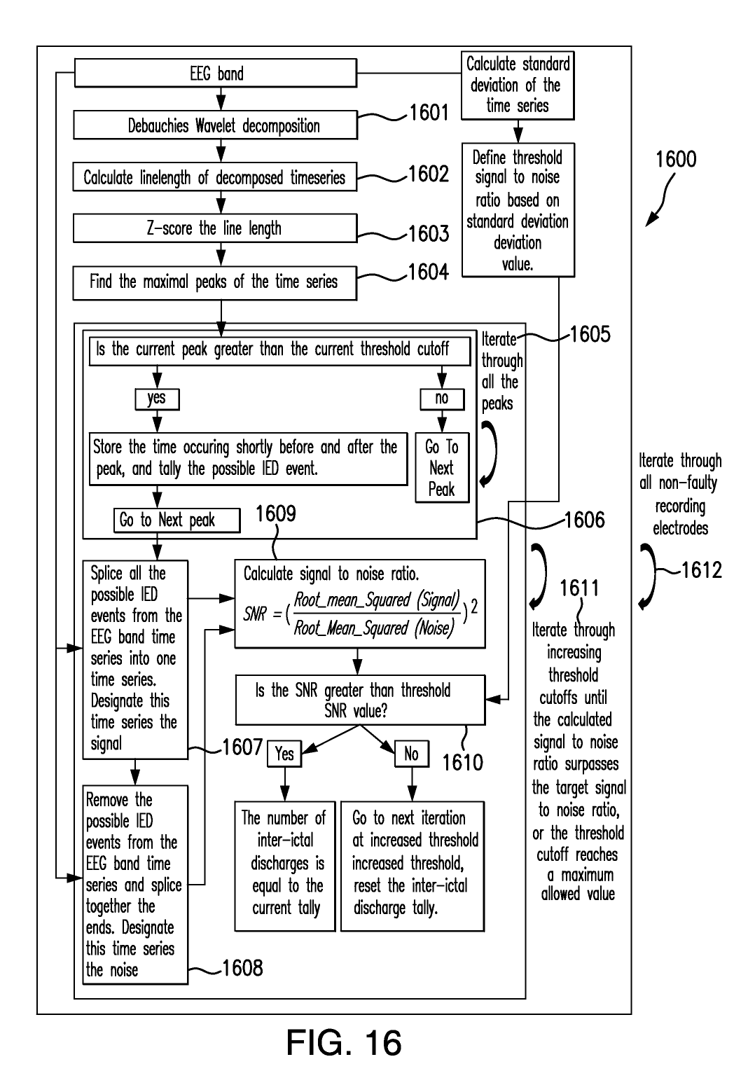

In addition to the detection of phase locked HFOs in the inter-ictal record, the detection of inter-ictal discharges can also be important for determination of epileptogenic brain. FIG. 15 shows, for the purpose of illustration and not limitation, inter-ictal discharges isolated from inter-ictal recording of an electrode adjacent to epileptogenic brain. These discharges were isolated using the method shown in FIG. 16.

FIG. 16 shows, for the purpose of illustration and not limitation, a method (1600) for isolating inter-ictal discharges from inter-ictal recordings. Inter-ictal discharge detection can be accomplished by performing a Debauchies wavelet decomposition of the EEG band pass filtered recording of the inter-ictal epoch using a Debauchies 4 wavelet at a level of 4 (1601). The line length of the decomposed time series can be calculated (1602) and normalized (1603), for example with a Z-score. A peak detection algorithm can be applied to the normalized time series and the time of the data points corresponding to each maximal peak can be stored in memory (1604). The method can include iterating through all the peaks in the normalized time series (1605) and determining if the normalized amplitude at each peak exceeds a valid peak threshold value (1606). If so, two time points shortly before and after the peak can be stored in memory, and the peak can be tallied as a possible inter-ictal discharge event. A time series (“signal”) can be created that consists of all the candidate inter-ictal discharge event in the inter-ictal EEG band pass filtered signal spliced together (1607). The time intervals of the candidate inter-ictal events in the inter-ictal EEG band pass filtered signal can be determined on the basis of the time locations of the peri-valid peaks in the normalized time series. Whereas, another time series can be created (“noise”) that consists of the inter-ictal EEG band pass filtered signal with the possible inter-ictal discharge events deleted and the open ends spliced together (1608). The SNR can be calculated using Equation 8 (1609).

$$SNR = {( \frac{Root\_Mean \_Squared ( Signal )}{Root\_Mean \_Squared ( Noise )} )}^{2} \qquad Equation 8$$

The calculated SNR can be compared to a threshold SNR value that can be a function of the standard deviation of the EEG band pass filtered recording of the inter-ictal epoch. If the calculated SNR exceeds the threshold SNR value, the number of inter-ictal discharges currently tallied can be set as the number of inter-ictal discharges detected in the record (1610). If the calculated SNR does not meet the threshold, the valid peak threshold value can be increased, and the number of inter-ictal discharges can be set to zero. The identification of valid peaks and calculation of the SNR can be repeated. The loop can be repeated until the calculated SNR is greater than the threshold value, or the valid peak threshold value reaches a predefined maximum (1611). In the latter case, the number of detected inter-ictal discharges can be designated as zero. The method can be repeated for all the recording sensors (1612).

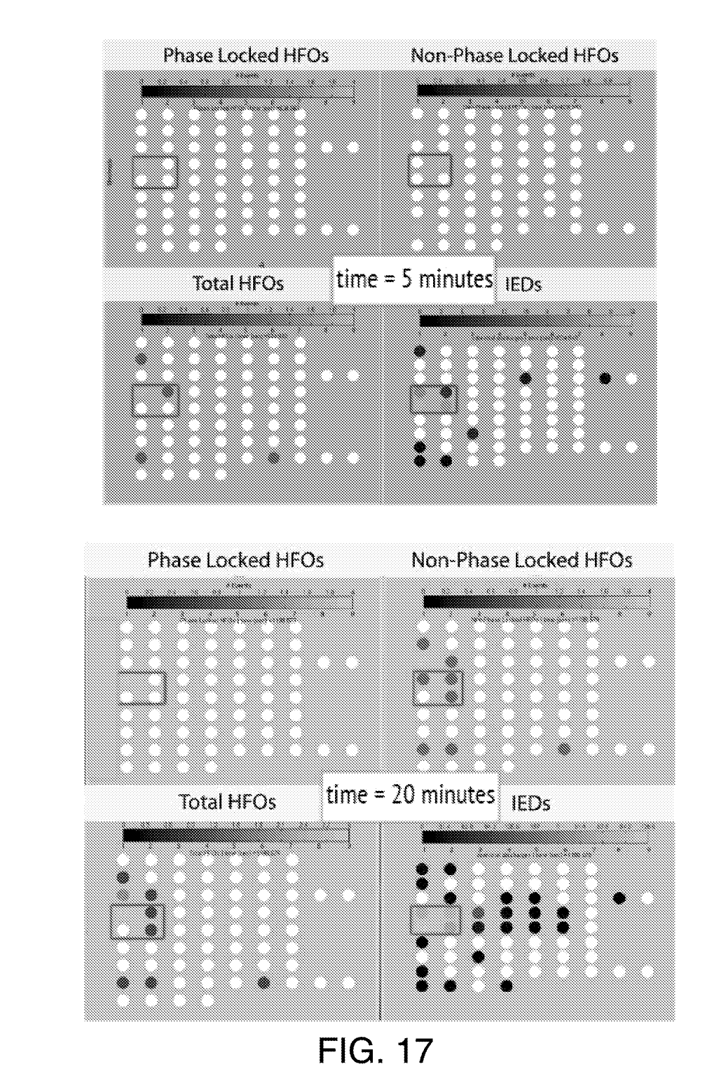

The methods presented in FIGS. 10, 11, 12, and 16 were tested in real-time during a recording of inter-ictal activity from intracranial electrodes in a patient. The results of the methods and device were updated every two minutes on the basis of repeating the algorithms described herein every two minutes on buffered segments of live intracranial brain recordings. FIG. 17 shows, for the purpose of illustration and not limitation, the location of the HFOs, PLHFOs, non-PLHFOs, and inter-ictal discharges after 5 minutes and 20 minutes of recording. The black square corresponds to the electrodes in the seizure onset zone where PLHFOs and inter-ictal discharges are over-represented.

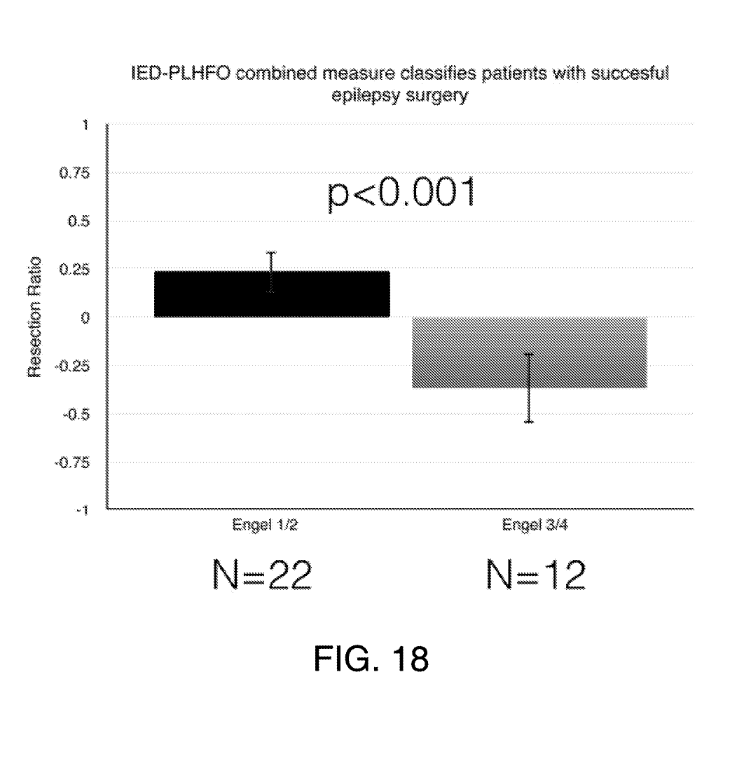

The IED-PLHFO metric, a biomarker of epileptogenecity, can be calculated on the basis of multiplication of the spatial maps of the relative number of inter-ictal discharges and the relative number of PLHFOs. Patients with successful epilepsy surgery had significantly more of IED-PLHFOs resected than patients with failed epilepsy surgery (as shown in FIG. 18). The methods described herein can be performed in real time on a computer system providing live and continuous data pertaining to the location of epileptic brain.

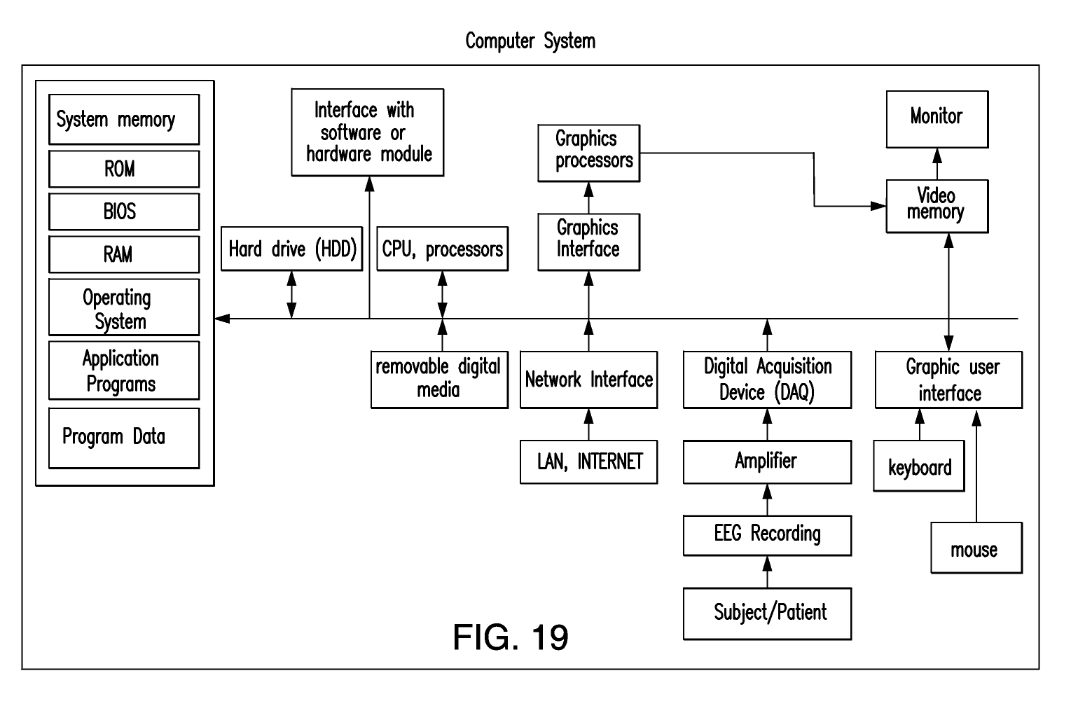

FIG. 19 shows, for the purpose of illustration and not limitation, a block diagram of an example computer system on which the methods described herein may be implemented as software or hardware. In these embodiments, each component can include a combination of hardware and software. One implementation can be to write source code that can be compiled into computer-readable instructions that can be processed by the central processing unit. The computer system can include input methods such as readable media, and data received over a local area network or the Internet, and data acquired in real-time from a data acquisition device connected to an amplifier receiving a signal from a patient or animal. The system memory can include read-only memory and random access memory. The system can include a basic input-output system that can transfer information between elements within the computer, and, for example, a hard disk drive. Commands can be entered into the computer using input devices, for example keyboard, mouse or other suitable devices. The results of the algorithms can be displayed on a graphic user interface. Commands entered into the computer can be used to interact with or modify the results obtained from the methods implemented on the computer system. Alternatively or additionally, the results of the methods can be delivered to another software module or hardware module. The computer system described herein can be implemented as a desktop, laptop, stand-alone device, or device implanted into the patient's or animal's body.

In certain embodiments, the methods and systems disclosed herein can be employed in supporting a therapeutic procedure based on the identified brain electrical activity displaying PLHFO. Such therapeutic procedures can include surgical resection of a portion of the brain, targeted gene therapy of a portion of the brain, and/or implanting a therapeutic device in the brain. In certain embodiments, the methods and systems disclosed herein can include identifying a neurological or psychiatric illness associated with the PLHFO. In certain embodiments, such neurological or psychiatric illnesses associated with the PLHFO include, but are not limited to, one or more structural lesions to the brain, such as brain tumors.

### Example 1

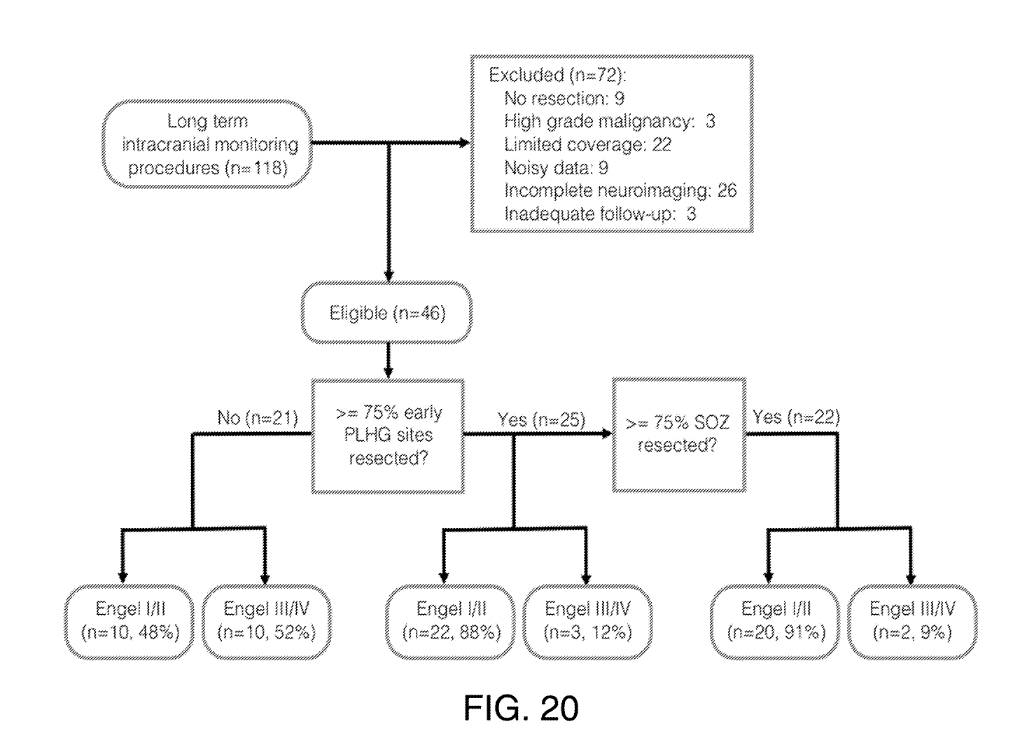

Methods: Data were obtained from consecutive epilepsy surgeries meeting study criteria (FIG. 20) at Columbia University Medical Center (CUMC, 2005-2012) and the National Hospital for Neurology and Neurosurgery in London (NHNN, 2011-2013). The study was approved by the Institutional Review Board at CUMC, and by the National Research Ethics Service at NHNN. Electrode configurations were customized for each patient and included subdural electrodes (3.0 mm diameter) with 0.5 or 1 cm center-to-center spacing, at times accompanied by depth (2.3 mm length) electrodes (Ad-Tech, Racine, Wis.). Data were recorded with standard clinical video EEG systems (XLTek at CUMC, Nicolet One at NHNN, Natus Medical Inc., Oakville, ON, Canada) sampled at 500 or 1000 Hz per channel, with bandpass filtering between 0.5 Hz and 1/4 sampling rate, 24/16 bit precision, and 0.31/0.15 μV resolution, respectively. Postoperative seizure outcome was classified using the Engel scale, with good outcomes defined as Engel class I or II and poor outcomes as class III or IV. The SOZ was determined by visual EEG review (typically 1-70 Hz bandpass filter, 10 seconds per screen) by the treating epileptologists at the time of the evaluation as the sites of the earliest departure from interictal patterns leading to sustained seizure activity, including rhythmic waveforms and spiking, and including rapid spread within one second. Although high frequency (>80 Hz) data were theoretically available at the time of surgery, it was not accessible in the clinical review software, nor was it part of standard clinical practice at either center during the study period.

EEG Signal analysis: The first three seizures recorded from each patient, including non-habitual seizures, were truncated to four minutes and analyzed. Subclinical seizures were excluded unless they were a previously-recognized seizure type. The PLHG measure was implemented as follows: Briefly, artifact was addressed either by excluding channels with excess 80-150 Hz noise to visual inspection, or removing noise using blind source separation with independent component analysis (EEGLAB, UCSD). Instantaneous high gamma (80-150 Hz, 500th order symmetric finite impulse response) amplitude, derived from the Hilbert transform and normalized to a 30 second preictal baseline, was weighted with the simultaneous phase-locking value computed from the low frequency (4-30 Hz) phase. Herald spikes, i.e. interictal-appearing discharges occurring immediately prior to the electrographic seizure onset, were excluded from both the seizure and the pre-ictal baseline. PLHG values were computed in 333 ms windows and averaged across 20 overlapping windows. The threshold for definition of a channel as a PLHG site was determined for each seizure independently, based on whether PLHG value distribution was bimodal, with clear separation between core and penumbral activity, or unimodal, where core sites were indicated by positive outliers. In the bimodal case, the threshold was defined as half the coefficient of variation less than the mean of the higher-valued distribution. In the unimodal case, the threshold was defined as three coefficients of variation over the mean. Line length (2-25 Hz, normalized to 30 second pre-ictal baseline with 2.5 SD threshold) was used as an objective measure approximating seizure spread as viewed in EEG. All calculations were fully automated and performed blinded to outcome using custom software (Matlab, Mathworks, Natick, Mass.).

Classification of electrodes: To define the resection boundaries and the position of implanted electrodes, pre-operative volumetric MRIs (1.5T or 3T) were co-registered to post-implantation CT scans and post-resection MRIs using the Advanced Normalization Tools, C3D (UPenn), FSL (Oxford, UK), and AMIRA (FEI Burlington, Mass.). Intra-operative photographs, detailed operative notes and EEG reports were used to confirm the final array placements. Following co-registration, 3D images were manually reviewed to identify the electrodes positioned within the resection boundaries.

Statistical Analysis: The locations of SOZ and PLHG electrodes were determined with respect to the resection cavity. The proportion of resected sites for each measure was then calculated. PLHG comparisons were calculated for “early” appearance (first four channels), “late” appearance (first eight channels), and for the entire seizure. The choice of the first four electrodes was based on the average size of the SOZ (4.8+/−0.4 channels). For SOZ, the resection ratio was calculated using all of the designated channels. Receiver operating characteristic (ROC) curves were constructed for the SOZ and early PLHG tests for 25%, 50%, 75%, and 100% resected cutoff values. True positives (sensitivity) were defined as the proportion of good outcomes above the test cutoff value and false positives (1—specificity) as the proportion of poor outcomes above the cutoff value. The quality of outcome classification was assessed using the area under the ROC curve (AUROC) and odds ratios. The Wilcoxon rank-sum test was used to compare measures including resection volumes, channel counts.

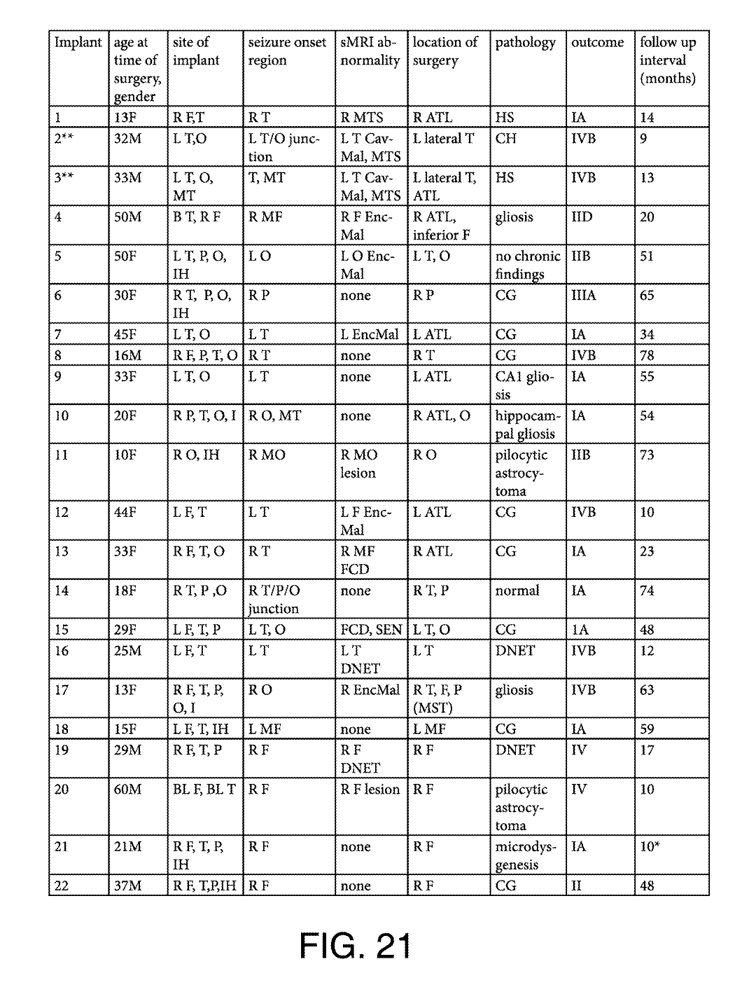

Results: Ninety-five consecutive epilepsy surgeries with chronic intracranial recordings were identified from CUMC and 33 from NHNN. Of these, 36 surgeries from CUMC and ten from NHNN were included (FIG. 20). In all, there were 102 seizures from 46 implants in 45 patients, with nine seizures in six patients lasting longer than four minutes. One patient underwent two implant procedures performed nine months apart (FIG. 21). Patient information for the study population: Outcomes are given according to Engel classification. One patient underwent two implants, indicated with asterisks. Pathology findings related to acute effects of surgical electrode implantation, e.g. reactive gliosis, are not included. Pathology reports describing only acute changes are listed as “no chronic findings”. Abbreviations include: L=left, R=right, B=bilateral, Post=posterior, F=frontal, T=temporal, P=parietal, 0=occipital, M=mesial, IH=interhemispheric, I=insular, C=cingulate, MTS=mesial temporal sclerosis, ATL=anteromesial temporal lobectomy, HS=hippocampal sclerosis, EncMal=encephalomalacia, FCD=focal cortical dysplasia, SEN=subependymal nodular heterotopia, DNET=dysembryoplastic neuroepithelial tumor, AVM=arteriovenous malformation, CH=cavernous hemangioma, CavMal=cavernous malformation, CG=Chaslin's marginal gliosis. MST=multiple subpial transections. * lost to follow up, ** sequential implants. Brain MRI scans for 24 patients (54%) lacked clearly localizing structural abnormalities, and 23 patients (52%) had nonspecific tissue pathology findings including gliosis. Good surgical outcomes were seen in 32 patients, and poor outcomes in 14 implantations in 13 patients. The rate of good outcomes was slightly better for patients with localizing lesions (73% vs. 67%). Excluding ten patients in whom volumetric data were not available, there was no difference in resection volumes between the outcome groups (26.0+/−4.3 cc3 (n=24 Engel I/II), 25.4+/−7.0 cc3 (n=12 Engel III/IV), p=0.67, Wilcoxon rank sum test). Mean follow-up time was 2.4+/−0.3 years, with a range of nine months-6.5 years (FIG. 21). Of the four patients with less than 12 months follow-up, all had poor (Engel IV) surgical outcomes, and two underwent subsequent epilepsy surgery procedures at the end of the follow-up period.

FIG. 7 illustrates the analysis in a patient (16) with recurrent post-operative seizures following a wide resection that included a well-defined left temporal lesion and the complete SOZ. PLHG was seen beginning 22 seconds after seizure onset in a small number of anterior temporal electrodes just anterior to the lesion and outside of the resection cavity. EEG traces from electrodes in the SOZ not meeting PLHG criteria (black disks) demonstrated an unequivocal EEG rhythm but no discernable high gamma bursting.

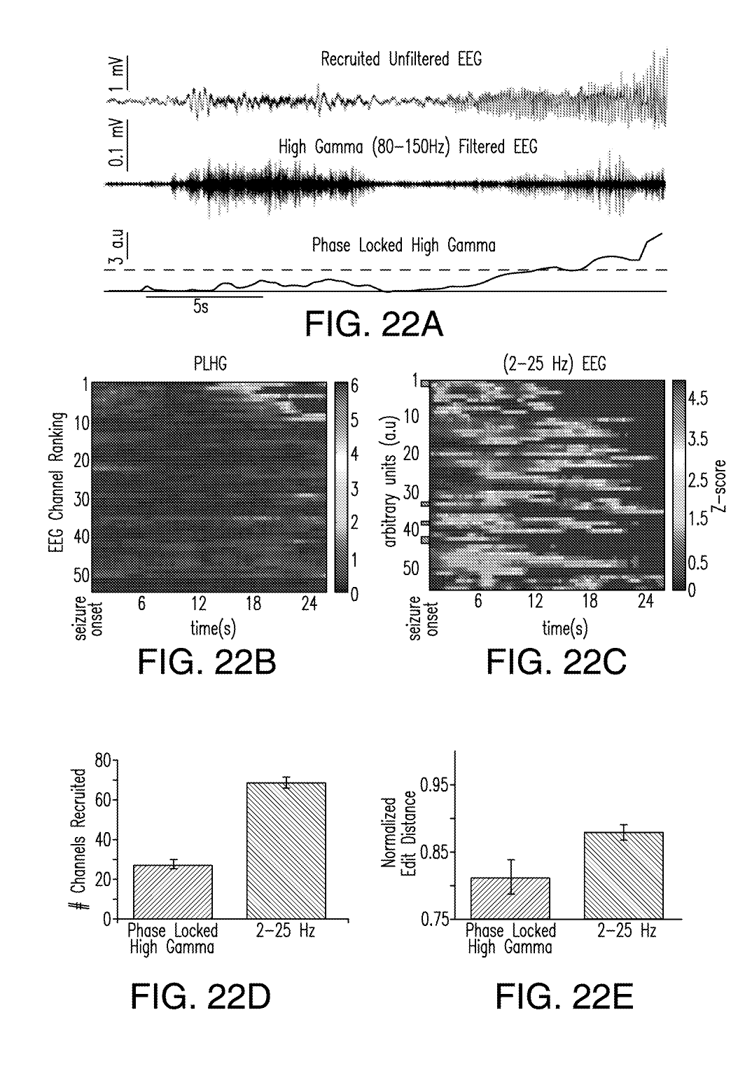

PLHG was identified in all but two patients, both of whom had Engel IV outcomes. Many channels exhibited increased high gamma amplitude (FIG. 22) without meeting PLHG criteria. The typical pattern was a brief attenuation in the high gamma filtered trace, followed by repetitive bursts aligned with the rhythmic waveforms seen in the raw EEG trace. PLHG values increased only with this latter amplitude-modulated pattern. The average time of earliest PLHG recruitment was 14.2+/−2.4 seconds after seizure onset.

The spatiotemporal evolution of sites defined as supra-threshold by the PLHG measure indicated a sharply demarcated region that expanded in tandem with the region demonstrating strong low-frequency discharges, but was always more limited in extent (FIG. 8). Across all seizures, the maximum number of PLHG electrodes was 21.0+/−1.6 by seizure termination (or four minutes, if seizure duration was longer), compared to 64.8+/−2.9 sites identified using line length (n=87 seizures, p<0.001, Wilcoxon rank sum test). In patients with multiple seizures, the PLHG recruitment sequence also demonstrated greater stereotypy among seizures in an individual patient than did line length (n=31, p<0.05, Wilcoxon rank-sum test). FIG. 8 illustrates the contrast between traditional visual EEG analysis (as indexed by the 2-25 Hz line length measure) and PLHG during seizure evolution, in a patient (42) with an Engel I outcome. At 12 seconds after seizure onset, PLHG was evident in depth electrode recordings from within the surgical margins (black arrows). By 21 seconds, PLHG had spread to neighboring subdural electrodes, also within the margins of the resection. In contrast, by 12 seconds, high amplitude EEG rhythms extended well outside the resection area.

We expected that resection of PLHG appearing early in the seizure would be superior to late-appearing PLHG as an outcome classifier (FIG. 9). Based on this observation, we chose to focus subsequent surgical outcome analysis on the early PLHG sites. Early PLHG was seen in an average of 45% of the SOZ channels. Both SOZ and early PLHG resection correlated with postoperative outcome classification. In patients with good outcomes, 91.5+/−2.6% of the SOZ was resected, while in patients with poor outcomes 65.0+/−10.5% of the SOZ was resected (n=46, p<0.05, Wilcoxon rank-sum test). Similarly, 72.7+/−5.1% of early PLHG sites were resected in patients with good outcomes, compared to 45.4+/−8.6% in patients with poor outcomes (n=46, p<0.01). To underscore the importance of the phase locking, we conducted a duplicate analysis using a measure based on high gamma (80-150 Hz) amplitude irrespective of phase, again focusing on early PLHG. The correlation with outcome classification was not statistically significant (p=0.06, Wilcoxon rank-sum test).

The SOZ was incompletely resected after 18 (39%) implant procedures. We constructed ROC curves using extent of resection of the SOZ (FIG. 9B) and early PLHG averaged across seizures for each patient. The AUROC values were 0.68 and 0.79, respectively. The relatively low specificity of the SOZ was especially notable: among implant procedures with poor outcomes, the SOZ was completely resected in six (46%), and 75% or more of the SOZ was resected in eight (62%). In contrast, 75% or more of early PLHG sites were resected in three (23%) cases with poor outcomes. The odds ratio for good outcome in the 35 patients with 75% or more of the SOZ resected was 5.3 [1.2-23.3], compared with 9.7 [2.3-41.5] for the 25 patients with resection of at least 75% of early PLHG sites. However, the difference between the two measures did not reach statistical significance.

We next asked whether sequential two-stage testing using both SOZ and early PLHG information would improve the accuracy of outcome classification. The two-stage AUROC improved to 0.86. Of the 22 patients meeting the 75% cutoff value for both SOZ and early PLHG, 91% had good outcomes, while poor outcomes were limited to just 9%. Fourteen of the patients (64%) became seizure free. Among patients with clearly localizing lesions, 91% (N=11) had good outcomes, vs. 92% (N=12) in non-lesional cases, with seizure-free rates of 64% and 58%, respectively. In contrast, 78% of the 36 cases with resection of at least 75% of the SOZ had good outcomes, and 53% became seizure free, with lower rates of good outcomes in the non-lesional group (79%/N=19 lesional cases vs. 60%/N=17 nonlesional). No difference was found for resection volumes between the 75% SOZ resection group (29.0+/−4.3 cc3, n=29) and the group with 75% of both SOZ and early PLHG electrode sites resected (28.8+/−5.1 cc3, n=20, Wilcoxon rank-sum test).

To provide further evidence that early PLHG sites can be the nidus for recurrent seizures, follow-up scalp and intracranial EEG recordings, available for 16 of the 23 patients with recurrent seizures (including Engel II outcomes), were evaluated. Early PLHG sites were left intact in 12 of these cases (75%). The follow-up studies demonstrated interictal discharges in eight patients and seizures in five patients whose localization was consistent (or not inconsistent) with the intact early PLHG sites. In three patients, recurrent interictal discharges and/or seizures localized to sites not sampled by the original intracranial electrodes. Notably, at least one of the seizures recorded intracranially prior to resection demonstrated no PLHG positive sites in two of these three patients. Patient 3 underwent a second implant that revealed PLHG sites adjacent to the edge of the prior resection, corresponding to an area where the first implant demonstrated a row of early PLHG sites just inside the resection boundary.

### Example 2

Methods: Data were obtained from an epilepsy surgery patient meeting study criteria. The electrode configuration included 9 depth (2.3 mm length) electrodes (Ad-Tech, Racine, Wis.). Data were recorded with a research clinical video EEG systems (Nihon Kohden Neurofax 1200, JE-120 research stream, Japan) sampled at 500 per channel, with bandpass filtering between 0.5 Hz and 1/2 the sampling rate, 16 bit precision. The live recordings from the research secondary stream were saved onto a ring buffer on a Dell Precision t3610 with 32 GB of memory (Dell Computer, Austin, Tx). Custom software (Matlab, Mathworks, Natick, Mass.) executed the algorithms described in FIG. 10, FIG. 11, FIG. 12, and FIG. 16 on 30-100 second chunks of the live intracranial EEG recording stored on the ring buffer. The statistical threshold for the HFO population vector strength cutoff was 0.25, and the given range of the phase angle was 90 degrees, the statistical cutoff for the Rayleigh test was p<0.05, and for the Rao test was of p<0.3. A color code was used to spatially graph the tally of inter-ictal discharges, total high frequency oscillations, phase locked high frequency oscillations, and non-phase locked high frequency oscillations. This graph was cumulatively updated each time the program processed the next chunk of live EEG stored in the ring buffer. The location of these events was compared to the location of the seizure onset zone determined by expert epileptologist review of a seizure captured earlier during the hospital stay.

Results: The location of the phase locked HFOs at 5 and 20 minutes into the recording were exclusively located in the seizure onset zone (FIG. 17). However, this was not for the case for non-phase locked HFOs. Many inter-ictal discharges also occurred in the seizure onset zone, however the inter-ictal discharges were widespread. The co-localization of inter-ictal discharges and phase locked HFOs correctly predicted the seizure onset zone.

While the disclosed subject matter is described herein in terms of certain exemplary embodiments, those skilled in the art will recognize that various modifications and improvements can be made to the disclosed subject matter without departing from the scope thereof. Moreover, although individual features of one embodiment of the disclosed subject matter can be discussed herein, or shown in the drawing of one of the embodiments and not in another embodiment, it should be apparent that individual features of one embodiment can be combined with one or more features of another embodiment or features from a plurality of embodiments. Thus, the foregoing description of specific embodiments of the disclosed subject matter has been presented for purposes of illustration and description. It is not intended to be exhaustive or to limit the disclosed subject matter to those embodiments disclosed.

1. A method for identifying brain electrical activity displaying phase-locked high-frequency oscillations (PLHFO), comprising:
receiving electrical signals from the brain;
filtering the electrical signals to produce a high frequency oscillation (HFO) data stream and a low-frequency data stream;
applying independent component analysis to the HFO data stream and removing noise from the HFO data stream;
transforming each of the HFO data stream and the low-frequency data stream to produce an HFO instantaneous amplitude and a low-frequency instantaneous phase;
normalizing the HFO instantaneous amplitude to produce a normalized HFO instantaneous amplitude;
transforming the normalized HFO instantaneous amplitude to produce an instantaneous phase of the normalized HFO instantaneous amplitude;
determining a PLHFO calculation based at least in part on the low-frequency instantaneous phase, the normalized HFO instantaneous amplitude, and the instantaneous phase of the normalized HFO instantaneous amplitude; and
determining that at least a portion of the electrical signals from the brain displaying PLHFO if the PLHFO calculation is above a threshold

2. A method for identifying brain electrical activity displaying phase-locked high-frequency oscillations (PLHFO), comprising:
receiving electrical signals from the brain;
filtering, transforming, and applying amplitude thresholds to the electrical signals to produce discrete high frequency oscillation (HFO) events comprised of the high frequency amplitude and low-frequency phase;
transforming each discrete HFO event and the low-frequency data to produce a discrete phasor based at least in part on an absolute amplitude of each discrete HFO event with respect to a corresponding phase of the low-frequency data;
optimizing to determine an optimal amplitude cutoff threshold for discrete phase locked HFO detection;
testing, using statistical tests, statistical significance of circular non-uniformity;
tallying a total number of PLHFOs if the circular non-uniformity shows statistical significance.

3. The method of claim 1, wherein receiving electrical signals further comprises recording electrical signals with an electroencephalogram (EEG).

4. The method of claim 2, wherein receiving electrical signals further comprises recording electrical signals with an electroencephalogram (EEG).

5. The method of claim 1, wherein recording occurs during a seizure.

6. The method of claim 2, wherein recording occurs between seizures.

7. The method of claim 2, wherein receiving electrical signals from the brain further comprises receiving recordings from a magnetoencephalography (MEG) device.

8. The method of claim 1, further comprising calculating the threshold using statistical methods that include unimodal and bimodal Gaussian mixture models.

9. The method of claim 1, further comprising supporting a therapeutic procedure based on the identified brain electrical activity displaying PLHFO.

10. The method of claim 9, wherein the therapeutic procedure comprises one of surgical resection of a portion of the brain or a lesion thereon, laser ablation of a portion of the brain or a lesion thereon, targeted gene therapy of a portion of the brain, and implanting a therapeutic device in the brain.

11. The method of claim 2, further comprising supporting a therapeutic procedure based on the identified brain electrical activity displaying PLHFO.

12. The method of claim 11, wherein the therapeutic procedure comprises one of surgical resection of a portion of the brain or a lesion thereon, laser ablation of a portion of the brain or a lesion thereon, targeted gene therapy of a portion of the brain, and implanting a therapeutic device in the brain.

13. The method of claim 1, further comprising identifying a neurological or psychiatric illness associated with the PLHFO, including a structural lesion to the brain such as a brain tumor.

14. The method of claim 2, further comprising identifying a neurological or psychiatric illness associated with the PLHFO, including a structural lesion to the brain such as a brain tumor.

15. The method of claim 1, wherein receiving electrical signals from the brain comprises receiving electrical signals from a plurality of recording electrodes.

16. The method of claim 15, further comprising mapping a portion of the electrical signals from the brain displaying PLHFO in space and time.

17. The method of claim 2, wherein receiving electrical signals from the brain comprises receiving electrical signals from a plurality of recording electrodes.

18. The method of claim 17, further comprising mapping a portion of the electrical signals from the brain displaying PLHFO in space and time.

19. The method of claim 1, wherein filtering the electrical signal includes applying a bandpass filter.

20. The method of claim 2, wherein filtering the electrical signal includes applying a bandpass filter.

21. The method of claim 1, wherein transforming the data streams comprises transforming the data streams with a Hilbert transform.

22. The method of claim 2, wherein transforming the data streams comprises transforming the data streams with a Hilbert transform.

23. The method of claim 1, wherein the method is automated.

24. The method of claim 2, wherein the method is automated.

25. The method of claim 2, further comprising detecting and tallying inter-ictal discharges based on a self-correcting signal splicing algorithm, and combining the tally with the tally of total number of PLHFOs.

26. The method of claim 2, wherein there statistical test is a Rayleigh's test.

27. The method of claim 2, wherein the statistical test is a Rao's test.

28. The method of claim 2, further comprising defining a location of epileptogenic brain based at least in part a spacial distribution of inter-ictal discharges and PLHFOs.

29. A system for identifying brain electrical activity displaying phase-locked high-frequency oscillations (PLHFO), comprising:
a data acquisition device for receiving electrical signals from the brain;
a memory storage system; and
a microprocessor configured to
filter the electrical signals to produce a high frequency oscillation (HFO) data stream and a low-frequency data stream;
apply independent component analysis to the HFO data stream and removing noise from the HFO data stream;
transform each of the HFO data stream and the low-frequency data stream to produce an HFO instantaneous amplitude and a low-frequency instantaneous phase;
normalize the HFO instantaneous amplitude to produce a normalized HFO instantaneous amplitude;
transform the normalized FIFO instantaneous amplitude to produce an instantaneous phase of the normalized HFO instantaneous amplitude;
determine a PLHFO calculation based at least in part on the low-frequency instantaneous phase, the normalized HFO instantaneous amplitude, and the instantaneous phase of the normalized HFO instantaneous amplitude;
determine that at least a portion of the electrical signals from the brain displaying PLHFO if the PLHFO calculation is above a threshold; and
provide information regarding the portion of the electrical signals from the brain displaying PLHFO to one of a clinician, secondary software, or device.

30. The system of claim 29, wherein the data acquisition device is configured to receive live electroencephalogram data over the Internet.

31. The system of claim 29, wherein the data acquisition device is configured to receive electrical signals from the brain saved on a storage device;

32. The system of claim 29, wherein the system comprises an implantable device.

33. A system for identifying brain electrical activity displaying phase-locked high-frequency oscillations (PLHFO), comprising:
a data acquisition device for receiving electrical signals from the brain;
a memory storage system; and
a microprocessor configured to
filter, transform, and apply amplitude thresholds to the electrical signals to produce discrete high frequency oscillation (HFO) events comprised of the high frequency amplitude and low-frequency phase;
transform each discrete HFO event and the low-frequency data to produce a discrete phasor based at least in part on an absolute amplitude of each discrete HFO event with respect to a corresponding phase of the low-frequency data;
optimize to determine an optimal amplitude cutoff threshold;
test, using statistical tests, statistical significance of circular non-uniformity;
tally a total number of PLHFOs if the circular non-uniformity shows statistical significance;
detect and tally inter-ictal discharges based on a self-correcting signal splicing algorithm,
provide information regarding the total number of PLHFO, and inter-ictal discharges to one of a clinician, secondary software, or device in real time.

34. The system of claim 33, wherein the data acquisition device is configured to receive live electroencephalogram data over the Internet.

35. The system of claim 33, wherein the data acquisition device is configured to receive electrical signals from the brain saved on a storage device;

36. The system of claim 33, wherein the system comprises an implantable device.
